(19) United States
(12) Patent Application Publication
(10) Pub. No.: US 2012/0232557 A1
(43) Pub. Date: Sep. 13, 2012

(54) METHOD FOR IMPROVING BLOOD FLOW IN BONE HEAD

(75) Inventors: Yuji Nakagawa, Ashigarakami-gun (JP); Yasushi Kinoshita, Ashigarakami-gun (JP); Yuichi Tada, Ashigarakami-gun (JP); Suguru Hata, Ashigarakami-gun (JP)

(73) Assignee: Terumo Kabushiki Kaisha, Shibuya-ku (JP)

(21) Appl. No.: 13/414,024

(22) Filed: Mar. 7, 2012

Related U.S. Application Data
(60) Provisional application No. 61/450,870, filed on Mar. 9, 2011.

(51) Int. Cl. A61B 17/16

(52) U.S. Cl. 606/80

(57) ABSTRACT

A method for improving the blood flow in the bone head, the method including the steps of extending a long tubular body, which has a cutting tool at its foreend, close to the entrance of the retinaculum artery and performing drilling on the bone head by using the cutting tool. This method makes it possible to improve the blood flow in the bone head with a minimum of burden on the patient.

### BACKGROUND OF THE INVENTION

1. Field of the Invention

The present invention relates to a method for improving the blood flow in the bone head. More particularly, the present invention relates to a method for improving the blood flow in the bone head (such as the head of the femur or humerus) suffering from osteonecrosis and also to a method for healing ischemic diseases leading to osteonecrosis.

2. Description of the Related Art

Osteonecrosis is one of the ischemic diseases that occurs in the head of the femur, the head of the humerus, or the condyle of the femur. Particularly, the osteonecrosis that occurs in the femoral head (the upper end of the femur held in the hip joint at the root of the leg) leading bony tissues to death, thereby causing depression or deformation to the joint surface. It reduces the blood flow in the femoral head and makes the bony tissues brittle due to necrosis. The progression of this symptom leads to the crushing of the femoral head and the osteoarthrosis of the hip joint, which brings about pains and gait disturbance, thereby greatly deteriorating the quality of life (QOL).

The cause of the osteonecrosis of the femoral head has not yet been completely cleared up; however, it appears without any cause or it appears relatively frequently in those who drink alcohol in large amounts or who experience systemic administration of steroid in large amounts. A possible cause for the osteonecrosis of the femoral head is the fracture in the neck of the femur which decreases the blood flow in the femoral head.

There are two methods for treating the osteonecrosis of the femoral head-conservative treatment and surgical treatment (operative treatment). The conservative treatment is applied to the case in which a good prognosis is anticipated by the size and position of necrosis or in which no necrosis has not yet appeared. It is practiced basically by non-weight bearing with a stick, and the patient is required to keep his weight, limit the distance of his walking, and refrain from carrying heavy loads. The patient with pains is given analgesic and antiphlogistic drugs. However, the conservative treatment is intended to relieve the symptom but is not intended to completely cure the osteonecrosis of the femoral head. In addition, because the conservative treatment is not fully expected to prevent the progress of crushing, the operative treatment is performed for conservation of the femoral head in the case where crushing is likely to progress. The operative treatment is accomplished by varus osteotomy (such as intertrochanteric curved varus osteotomy), rotation osteotomy of femur (in which the femoral head is turned forward or backward around the femoral neck such that the necrotized portion is relieved from loads and the healthy portion bears loads), and replacement of the crushed femoral head with an artificial head or replacement of the entire hip joint with an artificial hip joint. Unfortunately, the operative treatment mentioned above has to be performed under general anesthesia and needs the patient to be hospitalized for 5 to 7 days, depending on his age and conditions, followed by 6 weeks to 3 months for complete recovery. Moreover, the patient requires rehabilitation for a long period of time until he becomes capable of daily life without help.

As mentioned above, the osteonecrosis in the femoral head, once it appears, is hard to cure by conservative treatment and needs time and cost for surgical treatment. Thus, it causes a large damage to medical and social economy. Under these circumstances, early diagnosis and early treatment are desirable but there has been no effective method for treatment to prevent the progression of the disease.

### SUMMARY OF THE INVENTION

It is an object of the present invention to provide a method for improving the blood flow in the bone head, especially the femoral head with a minimum invasion on the patient.

It is another object of the present invention to provide a method for improving the blood flow in the femoral head, thereby treating the ischemic disease that causes the osteonecrosis in the femoral head.

According to its one embodiment, the present invention provides a method for improving the blood flow in the bone head, the method including the steps of extending a long tubular body, which has a cutting tool at its foreend, close to the entrance of the retinaculum artery and performing drilling on the bone head by using the cutting tool.

The method according to the present invention improves the blood flow in the head of the bone, particularly the head of the femur, with a minimum burden on the patient.

The above and other objects, features and advantages of the present invention will become clear from the following description of the preferred embodiments.

### BRIEF DESCRIPTION OF THE DRAWINGS

FIGS. 1A to 1F are diagrams illustrating the steps to be carried out in one way of the method according to the present invention;

FIGS. 2A to 2F are diagrams illustrating the steps to be carried out in another way of the method according to the present invention;

FIG. 3 is a schematic sectional view showing the long tubular body provided with a cutting tool (drill) at the foreend thereof, which is used for the first preferred embodiment of the present invention;

FIG. 4A is a schematic sectional view showing the long tubular body provided with a cutting tool (functional member) at the foreend thereof, which is used for the second preferred embodiment of the present invention;

FIGS. 4B to 4G are side views illustrating the structure of the cutting tool (functional member) attached to the long tubular body shown in FIG. 4A;

FIG. 5 is a schematic sectional view showing the long tubular body provided with a cutting tool (end effector) at the foreend thereof, which is used for the third preferred embodiment of the present invention;

FIG. 6 is a schematic sectional view showing the long tubular body provided with a cutting tool (rotary member) at the foreend thereof, which is used for the third preferred embodiment of the present invention; and

FIG. 7 is a schematic sectional view showing the long tubular body provided with a cutting tool (screw) at the foreend thereof, which is used for the third preferred embodiment of the present invention.

### DETAILED DESCRIPTION OF THE PREFERRED EMBODIMENTS

The present invention provides a method for improving the blood flow in the bone head, the method including (i) a step of extending a long tubular body, which has a cutting tool at its foreend, close to the entrance of the retinaculum artery, and (ii) a step of performing drilling on the bone head by using the cutting tool. The method of the present invention is applied to the femoral head, for example, in such a way that a long tubular body having a cutting tool at its foreend is inserted close to the entrance of the retinaculum artery that extends from the circumflex artery to the femoral head and the cutting tool drills at least one perfusion passage into the spongy bone of the femoral head beyond the epiphysis line, so as to promote the blood flow into the bone head, particularly the spongy bone of the bone head. The term “bone head” used in the present invention implies the spongy bone that forms the joint at the end of the long truncal bone. To be more specific, the bone head unrestrictedly includes the head of the femur, the head of the humerus, the condyle of the femur, the condyle of the humerus, the condyle of the shin bone, and the medial malleolus of the shin bone.

As mentioned above, conservative treatment involves difficulties in completely healing the osteonecrosis of the femoral head and is incapable of effectively preventing the progression of crushing, and surgical treatment has been the last resort. Unfortunately, surgical treatment for the osteonecrosis of the femoral head is greatly invasive on the patient in itself and requires extended rehabilitation that follows the operation.

The method according to the present invention entirely differs from the conventional one in that it employs a long tubular body (such as catheter, wire, and endoscope) having a cutting tool at its foreend, thereby forming at least one perfusion passage in the bone head. The perfusion passage permits blood to flow into it from the retinaculum artery and the circumflex artery which surrounds it. At this time, the angiogenesis factor (stem cells derived from the bone marrow) is also released into the perfusion passage. Such blood also contains the angiogenesis factor (stem cells derived from the bone marrow), which helps form blood vessels (capillary vessels) in the perfusion passage. This is the reason for improvement in the blood flow (or prevention of ischemia) in the bone head. The method according to the present invention does not need surgical treatment and hence is low-invasive on the patient. In addition, the method according to the present invention is effective particularly for patients in the early stage who show the symptom of osteonecrosis in the bone head before the bone head suffers from crushing (white asking).

The preferred embodiments of the present invention will be described below with reference to the accompanying drawings. The following detailed description is concerned with the method for improving the blood flow in the bone head (femoral head) suffering from the osteonecrosis of the femoral head, or the method for treatment of the ischemic disease, such as the osteonecrosis in the femoral head. The present invention is not restricted in its scope to the embodiments mentioned below, but it can also be applied to improvement in blood flow in other parts, such as the head of the humerus and the condyle of the femur, or to treatment of the ischemic disease, such as the osteonecrosis in the bone head.

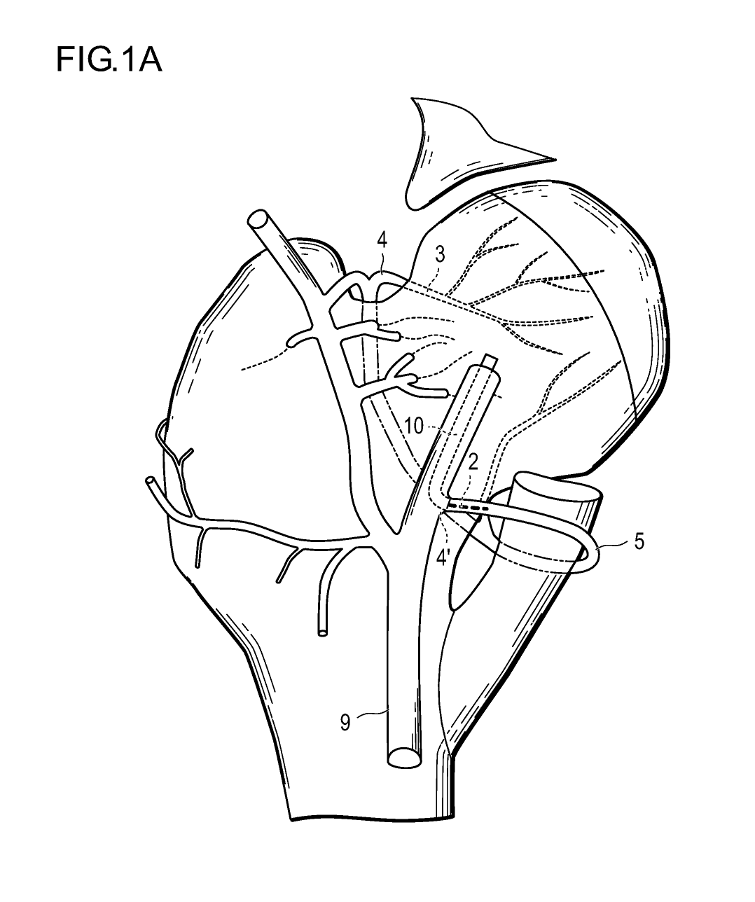

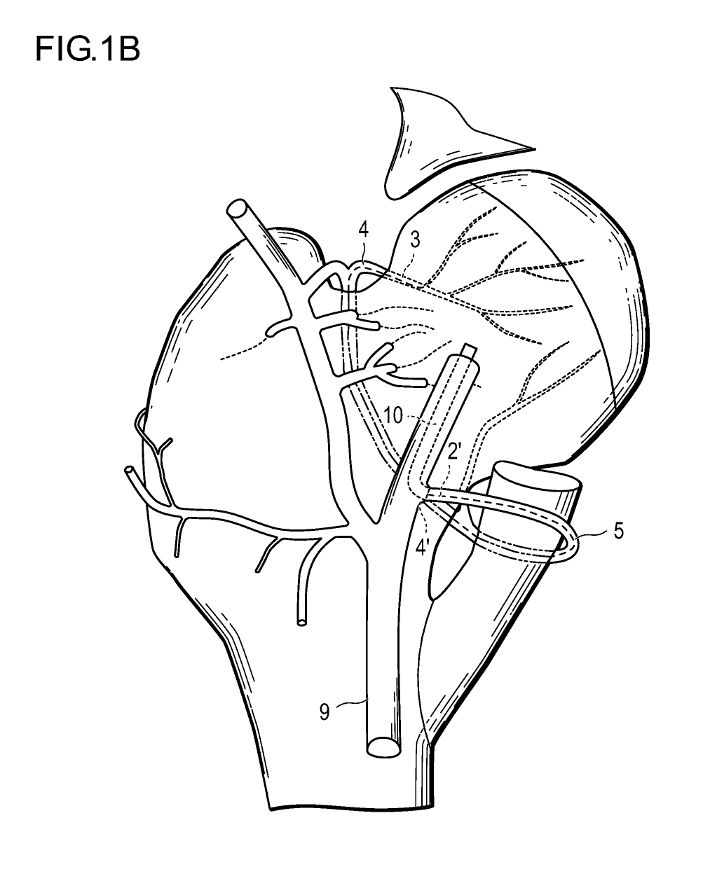

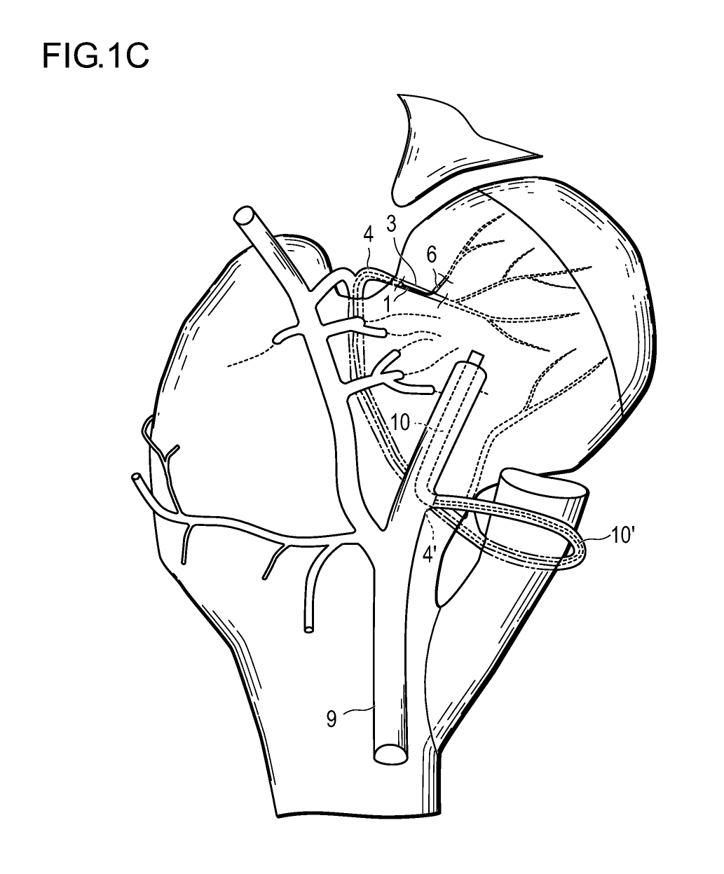

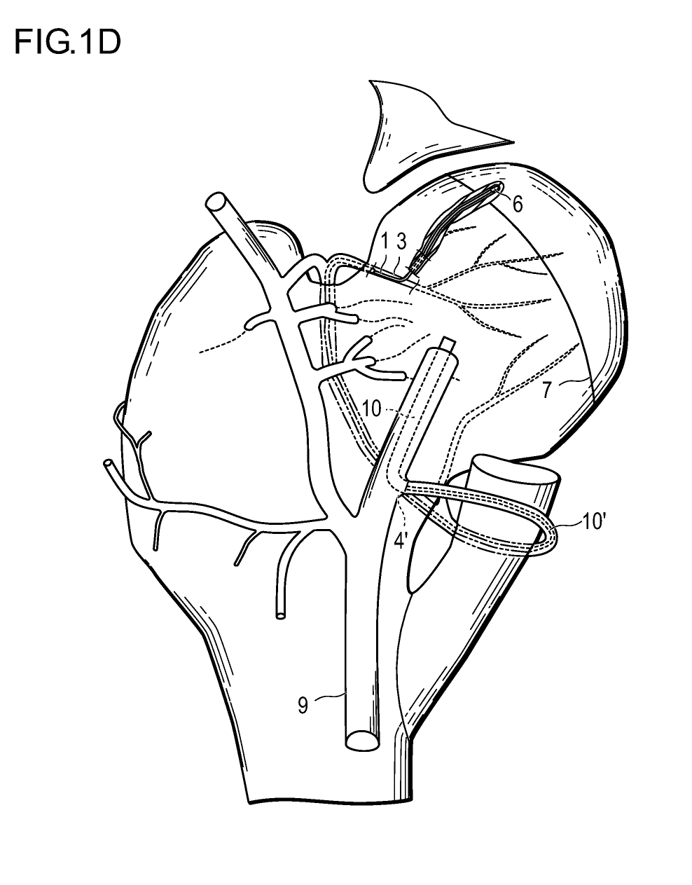

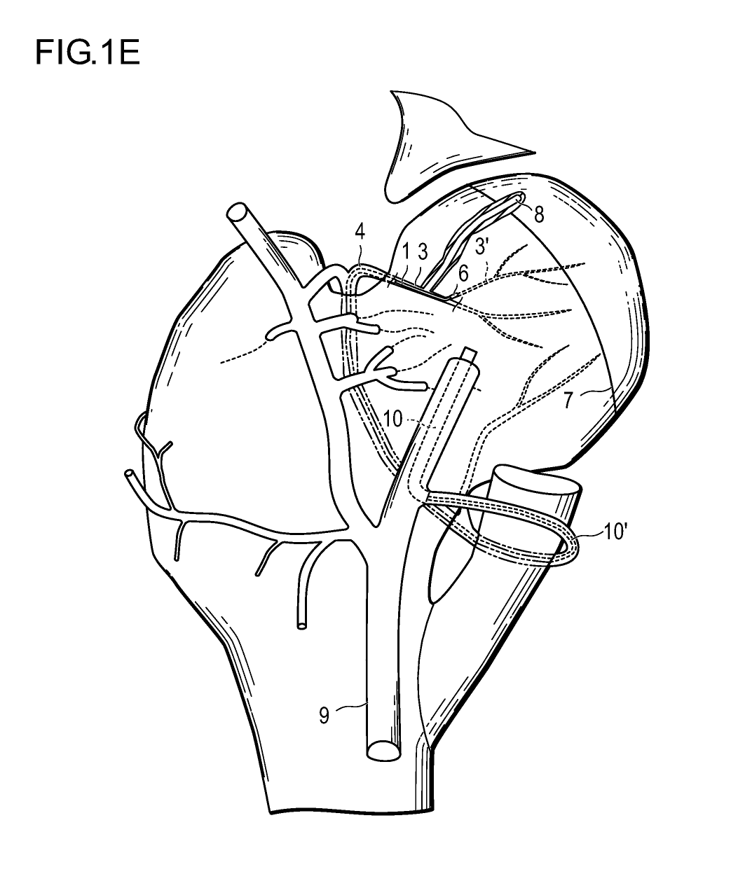

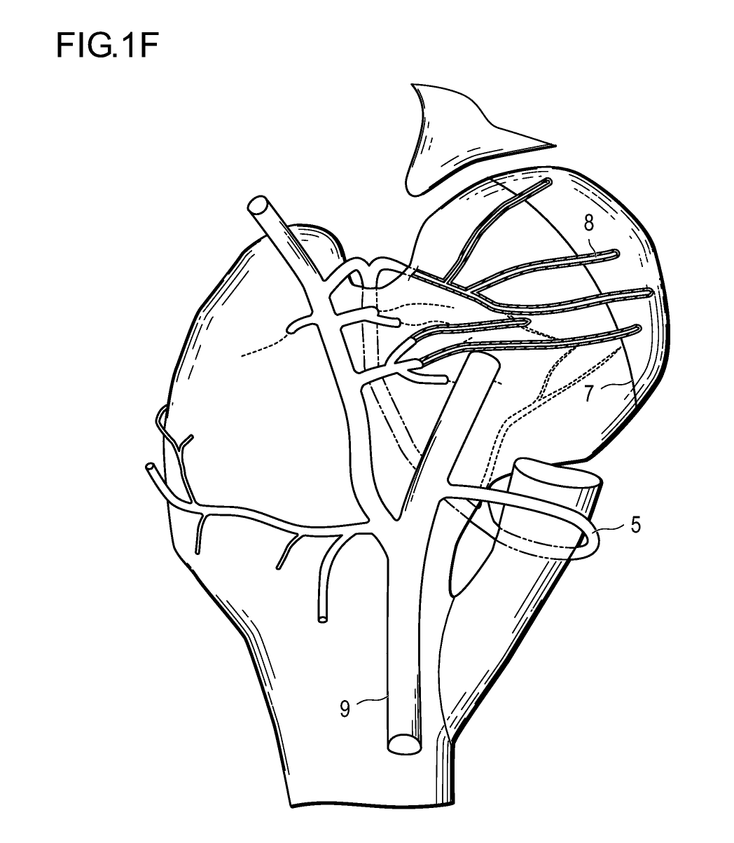

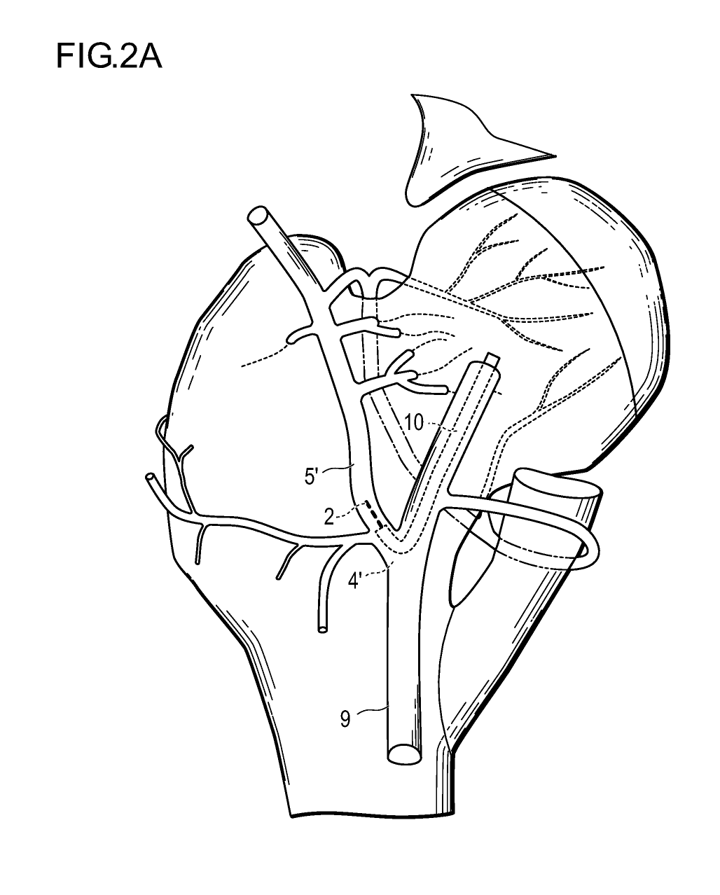

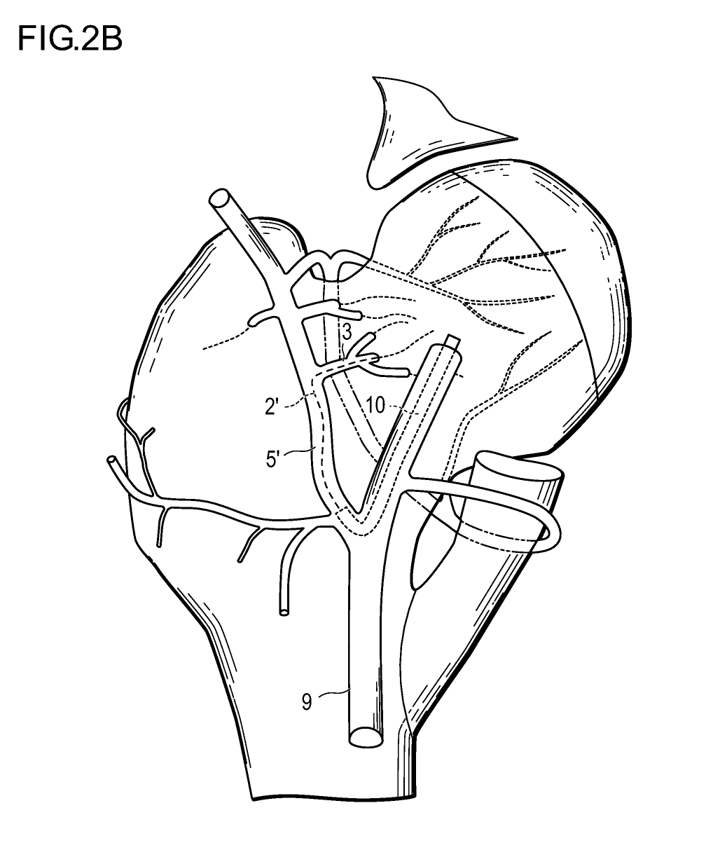

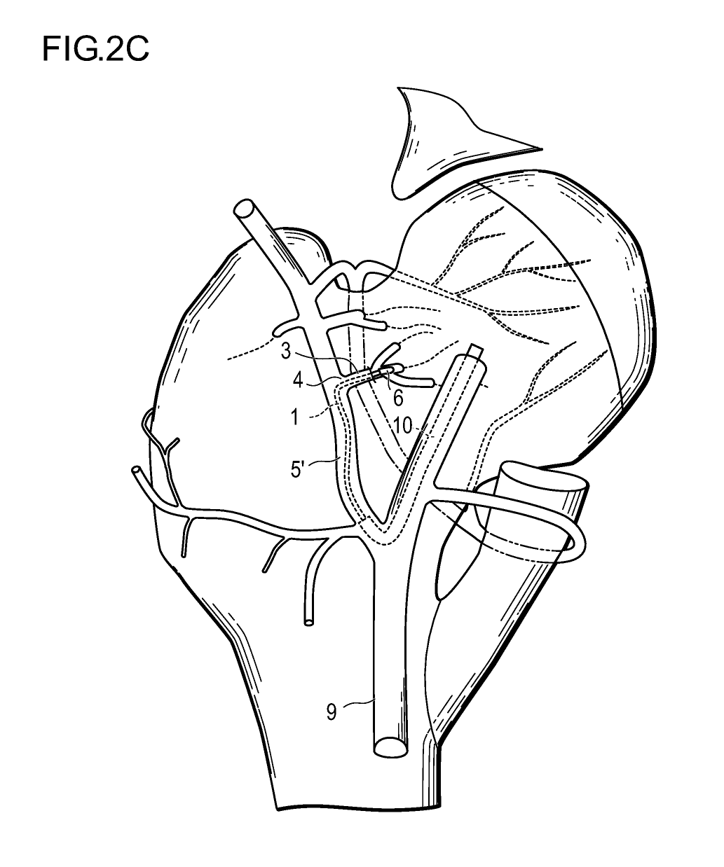

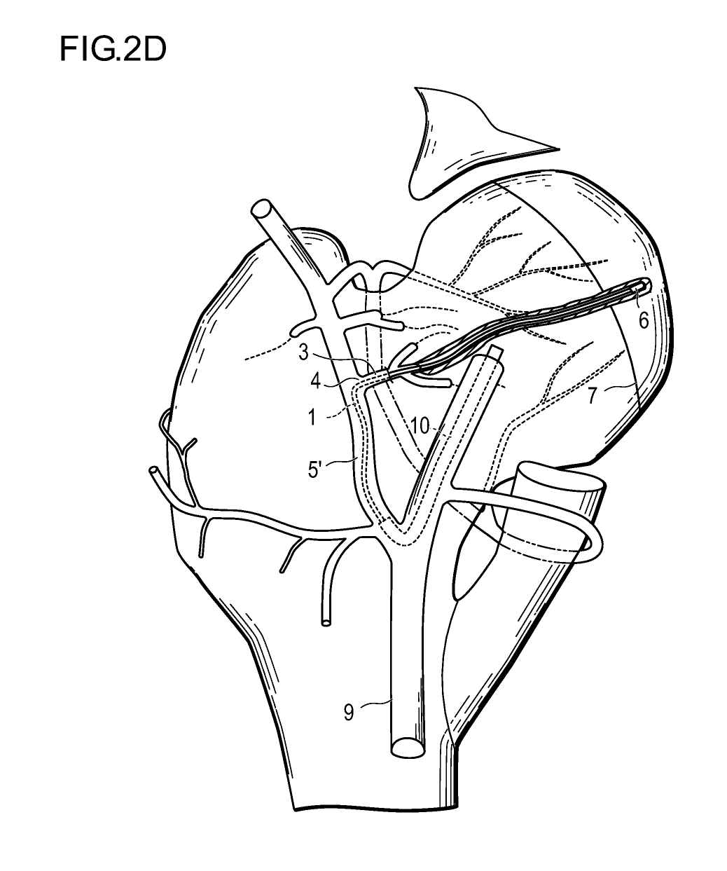

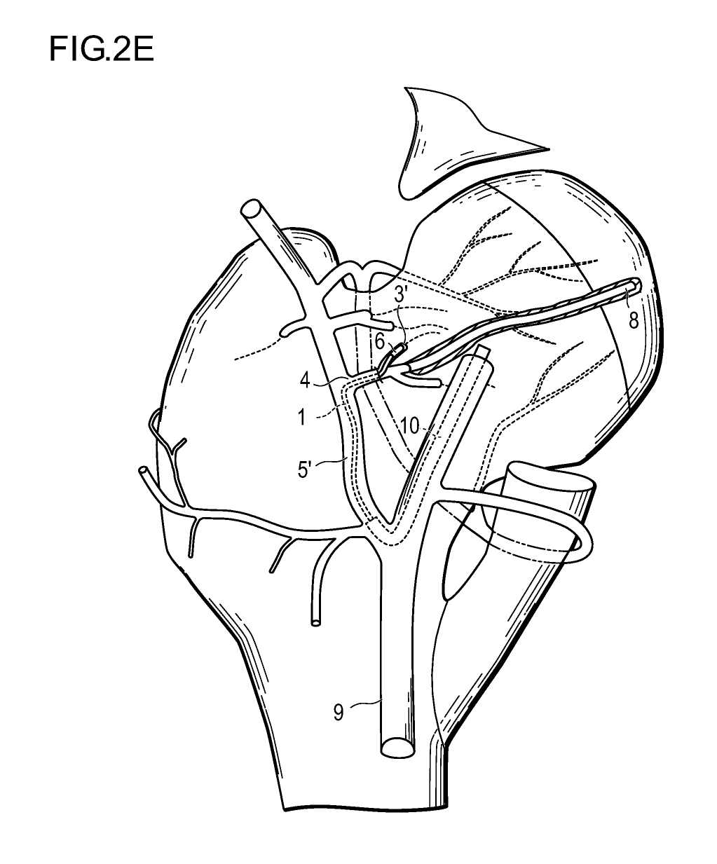

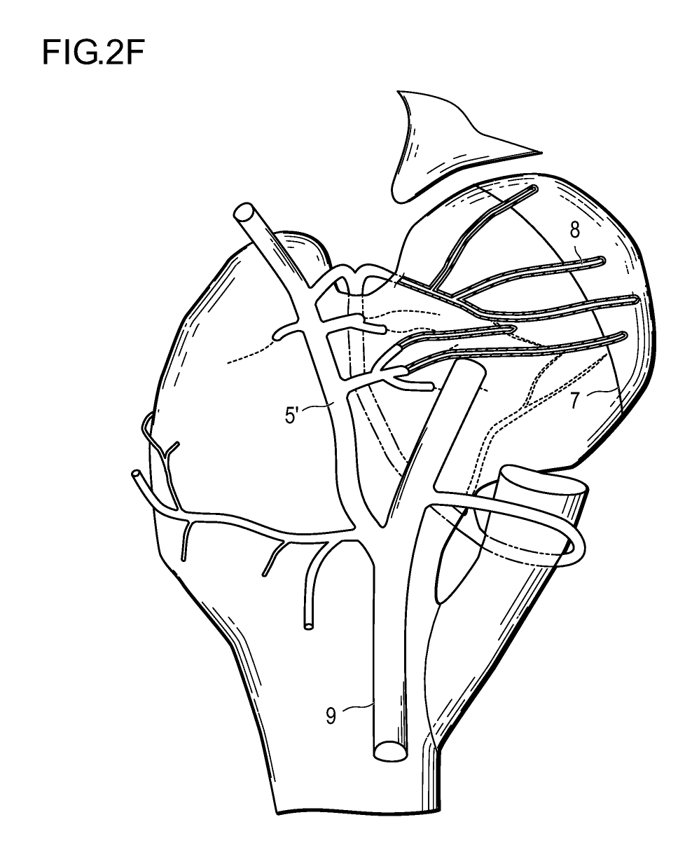

The method according to the present invention consists of two steps as shown in FIGS. 1 and 2.

Step (i) is intended to extend a long tubular body, which has a cutting tool at its foreend, close to the entrance 4 of the retinaculum artery 3. To be more specific, Step (i) is intended to advance the guide wire 2, the guiding catheter 10, the guide wire 2′ (thinner than the guide wire 2), and the microcatheter 10′ sequentially through the blood vessel and finally place the long tubular body 1 (having the cutting tool 6 at its foreend) in the retinaculum artery 3 through the microcatheter 10′, as shown in FIGS. 1A to 1C. Alternatively, Step (i) is intended to advance the guide wire 2, the guiding catheter 10, and the guide wire 2′ (thinner than the guide wire 2) sequentially through the blood vessel and finally place the long tubular body 1 (having the cutting tool 6 at its foreend) in the retinaculum artery 3 through the guide wire 2′, as shown in FIGS. 2A to 2C. Incidentally, it is desirable to perform X-ray or MRI examination on the patient suffering from femoral head osteonecrosis, thereby defining the extent of necrosis, prior to Step (i) mentioned above in order to specify the part that needs improvement in blood flow (or treatment for ischemia). In addition, it is also desirable to perform antithrombotic treatment prior to Step (i), because antithrombotic treatment permits blood to flow easily into the perfusion passage to be formed in Step (ii) that follows and also prevents the formation of thrombus after blood begins to flow (which is expected to promote the formation of new blood vessels). The antithrombotic treatment should be performed 1 to 48 hours before the start of Step (i). The length of time should be properly adjusted according to the package insert of the drug to be used. Incidentally, the antithrombotic treatment should be performed in such a way not to increase side effect, such as bleeding. The antithrombotic treatment that is performed at this timing permits sufficient blood to flow into the perfusion passage to be formed in Step (ii), thereby retarding the formation of thrombi in the drilled part in the bone and hence promoting the formation of new blood vessels. The method for antithrombotic treatment is not specifically restricted; any known one may be used as such or with adequate modifications. Typical methods include administration of any known antiplatelet agent, such as cyclooxygenase (COX-1) inhibitor, prostaglandin, thromboxiane synthesizing enzyme inhibitor, thienopyridine derivative, phosphodiesterase 3 (PDE3) inhibitor, 5-serotonin receptor 2 antagonist, GP IIb/IIIa inhibitor, and clopidogrel; and anticoagulant, such as vitamin K dependent blood coagulation factor synthesis inhibitor and heparin. The antiplatelet agent, such as clopidogrel, should preferably be orally administered in combination with aspirin (81 to 100 mg/kg weight/day).

In Step (i), no specific restrictions are imposed on the method of placing (or introducing) the long tubular body, which has a cutting tool at its foreend, at (or close to) the entrance of the retinaculum artery. For example, the long tubular body may be introduced into the femoral artery, brachial artery, or radial artery, which is the same position as selected for the ordinary catheter. It is desirable to insert the long tubular body from the position close to the target of drilling in consideration of burdens for the patient. An example of such positions for insertion of the long tubular body is the femoral artery for improvement of the blood flow in the femoral head or the thigh opposite to the one which has the lesion in consideration of easy operation. The insertion of the long tubular body to the desired part may be facilitated with the help of another guide wire, guiding catheter, and microcatheter. According to one embodiment, this operation may be accomplished in the following way. First, a sheath is detained in the femoral artery in the thigh opposite to the one which has the lesion. Next, this sheath permits the guide wire 2 (0.035 inches in diameter, for example) to advance into the femoral artery (not shown) in the lesion under X-ray radioscopy and then into the inner femoral circumflex artery 5 through the deep artery of the thigh 9. Then, the foreend of the guiding catheter 10 (4 Fr, for example), with its one lumen slipped on the guide wire 2, is engaged (placed) near the branch point (entrance) 4′ of the inner femoral circumflex artery 5 and the deep artery of the thigh 9, as shown in FIG. 1A. Then, this guide wire 2 is withdrawn and another thinner guide wire 2′ (0.014 inches in diameter, for example) is inserted into the guiding catheter 10. This guide wire 2′ is advanced into the retinaculum artery 3 through the inner femoral circumflex artery 5, as shown in FIG. 1B. The foreend of the microcatheter 10′ (2.2 Fr, for example), with its one lumen slipped on the guide wire 2′, is introduced (or placed) inside the retinaculum artery with the help of the guide wire 2′, as shown in FIG. 1C. Alternatively, the foreend of the microcatheter 10′ (2.2 Fr, for example), with its one lumen slipped on the guide wire 2′, is introduced (or placed) inside the retinaculum artery through the guiding catheter 10. In this way, the microcatheter 10′ is firmly fixed inside the retinaculum artery which is away from the entrance of the retinaculum artery. This ensures the drilling of the bone head in the ensuing step with the help of the long tubular body having a cutting tool at its foreend. Alternatively, the microcatheter is placed inside the retinaculum artery or near the entrance of the retinaculum artery by using the microcatheter 10′ having an expanding part (such as balloon) at its foreend, and then the microcatheter is fixed in the artery by means of the expanding part which has been expanded. The operation in this manner permits the microcatheter 10′ to be firmly fixed inside the retinaculum artery which is away from the entrance of the retinaculum, and this ensures the drilling of the bone head in the ensuing step with the help of the long tubular body having a cutting tool at its foreend. Subsequently, the guide wire 2′ is withdrawn and the long tubular body 1 having a cutting tool 6 at its foreend is inserted into the lumen of the microcatheter 10′ and the cutting tool 6 is introduced into the retinaculum artery 3, as shown in FIG. 1C. According to another embodiment, a sheath is detained in the femoral artery in the thigh opposite to the one which has the lesion. Next, this sheath permits the guide wire 2 to advance into the femoral artery (not shown) in the lesion under X-ray radioscopy and then into the point slightly away from the branch point (entrance) of the deep artery of the thigh 9 and the outer femoral circumflex artery 5′. Then, the foreend of the guiding catheter 10, with its one lumen slipped on the guide wire 2, is engaged (placed) near the branch point (entrance) 4′ of the outer femoral circumflex artery 5 and the deep artery of the thigh 9, as shown in FIG. 2A. Then, this guide wire 2 is withdrawn and another thinner guide wire 2′ is inserted into the guiding catheter 10. This guide wire 2′ is advanced into the retinaculum artery 3 through the outer femoral circumflex artery 5′, as shown in FIG. 2B. The long tubular body 1 having the cutting tool 6 at its foreend is advanced into the retinaculum artery 3 with the help of the guide wire 2′, as shown in FIG. 2C. Finally, the guide wire 2′ is withdrawn. The operation in this manner permits the foreend (cutting part) of the long tubular body to be placed (through the blood vessel) inside the retinaculum artery extending into the bone head where drilling starts. In the foregoing embodiments, the guiding catheter or the long tubular body may have a balloon attached to its foreend. The balloon allows easy introduction of the catheter or long tubular body into the desired position with the help of the blood flow.

The long tubular body to be used in the present invention is not specifically restricted so long as it has a cutting tool at its foreend. Its selection depends on the diameter or branch type of the artery into which it is inserted. The long tubular body having a cutting tool is usually used in combination with a guide wire and guiding catheter for its introduction to the vicinity of the entrance of the retinaculum artery and means for revolving the cutting tool. To be more specific, it may be converted from medical instruments, with or without modifications, such as endoscopes and catheters to pick up part of living tissues, such as endoscopes and catheters to destroy thrombi formed in the blood vessel, thereby allowing the blood flow to restart, or such as catheters (provided with a rotablator for atheroma excising) which are used for treatment of coronary artery occlusion. Some examples of such medical instruments are listed below.

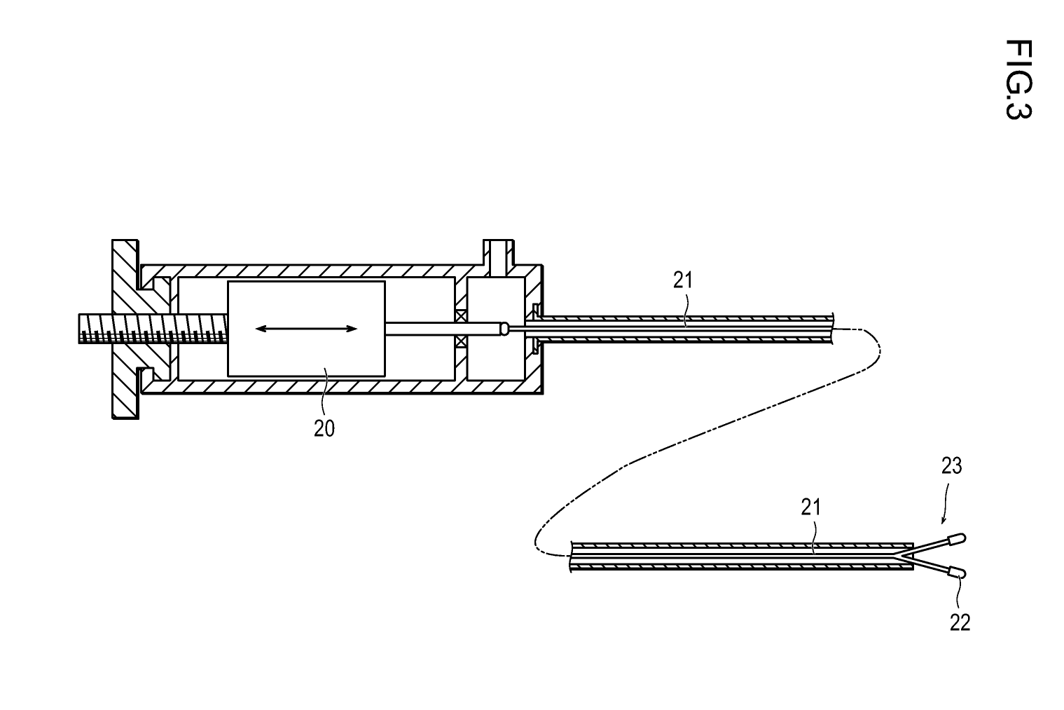

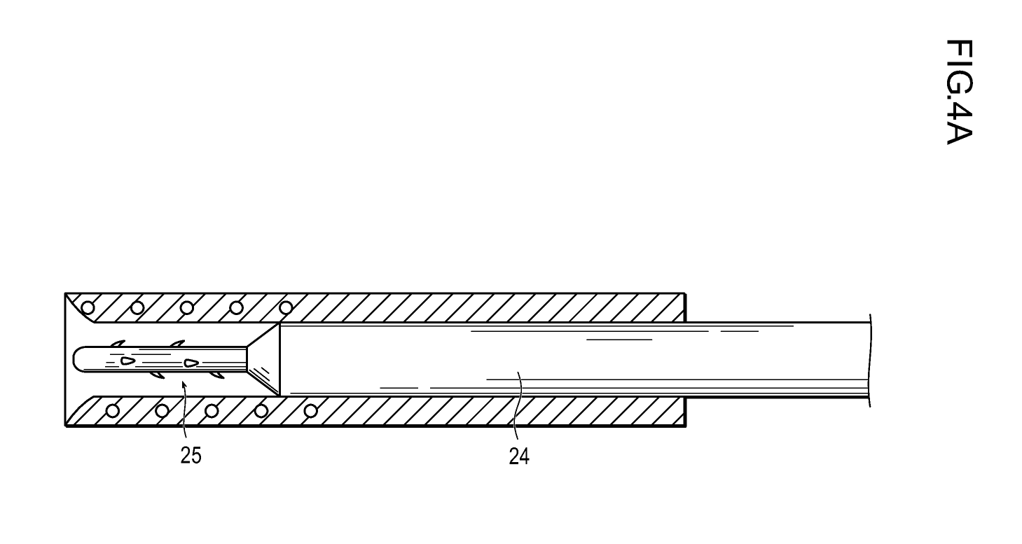

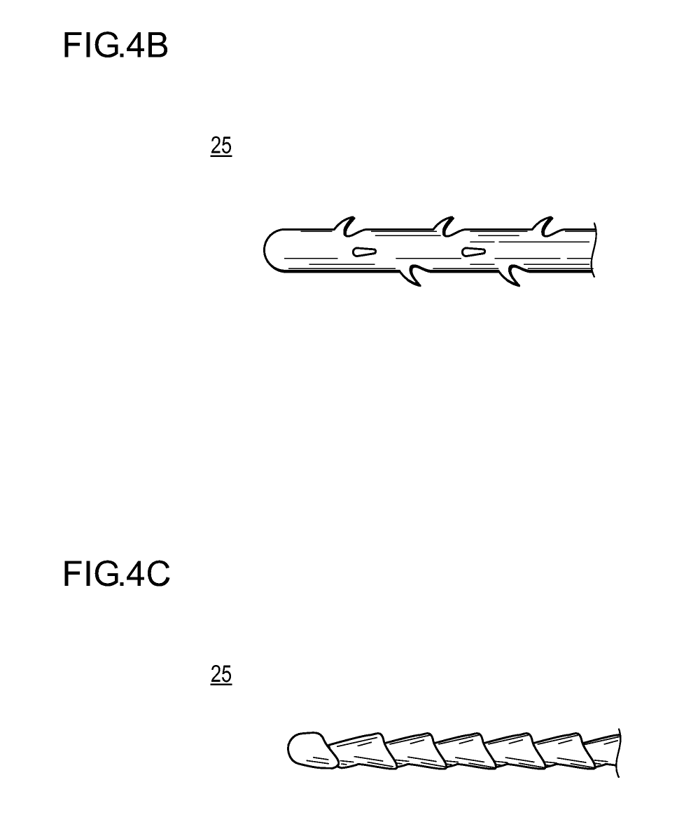

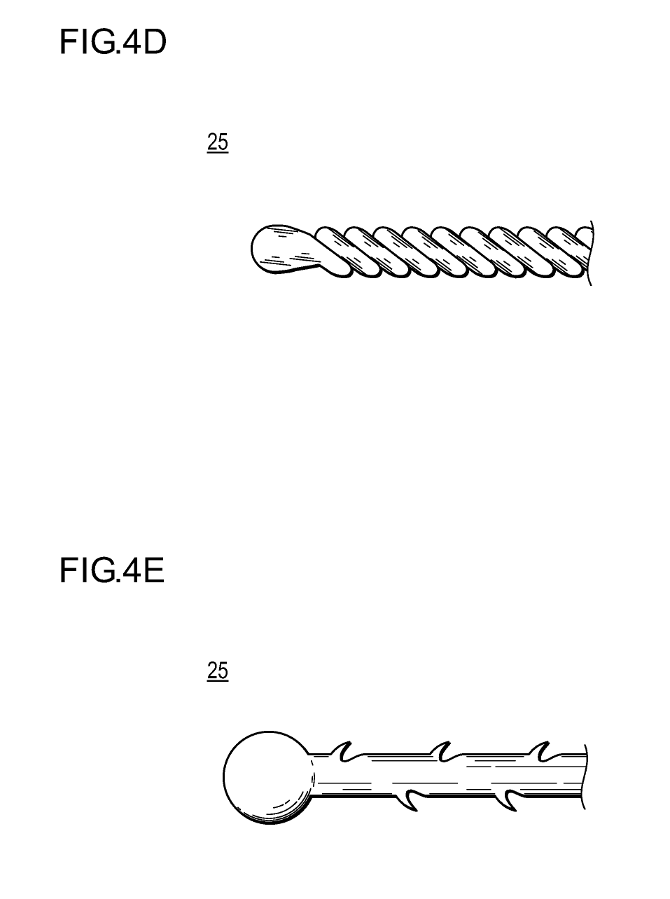

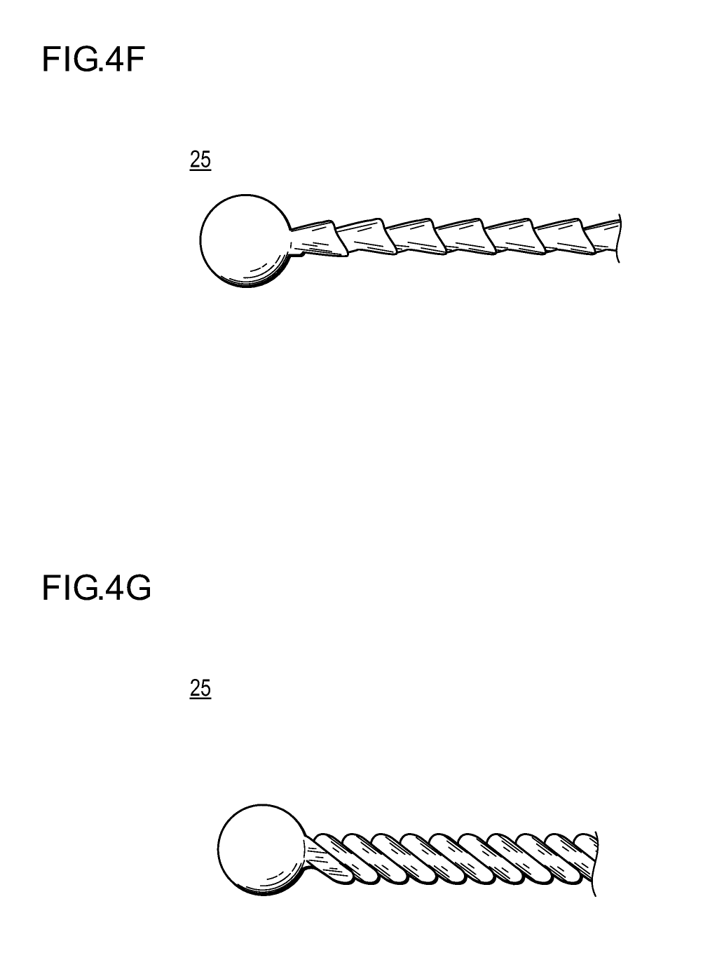

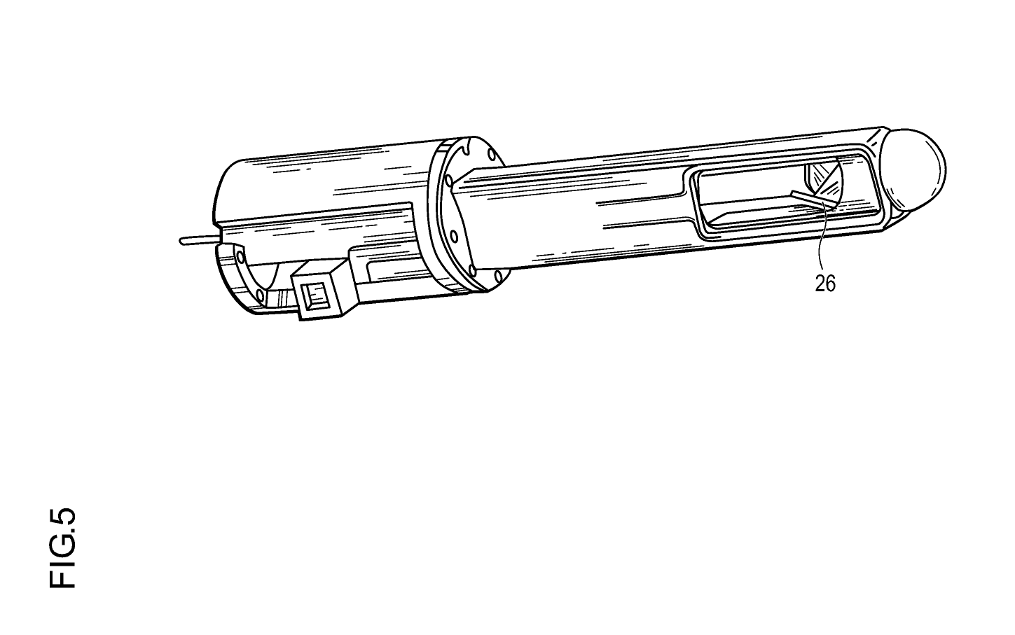

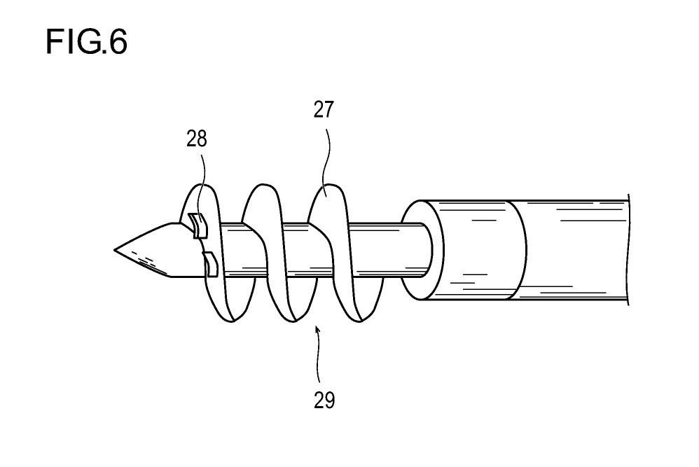

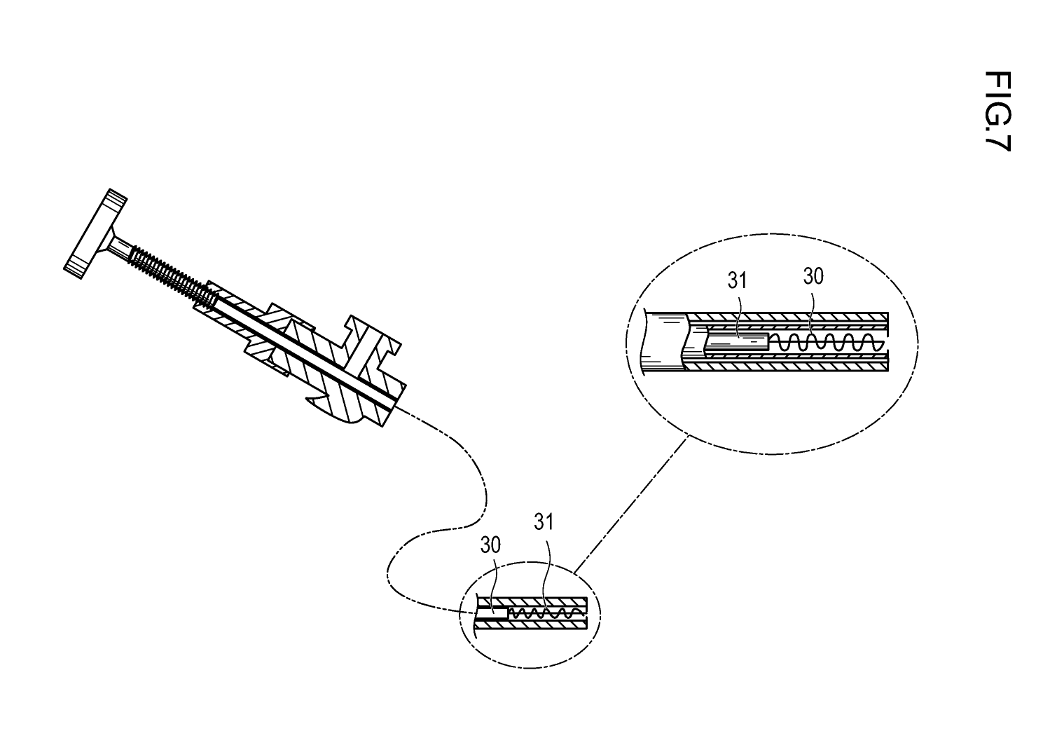

(a) The one shown in FIG. 3 which is composed of a wire 21 and a drill 23 (as a cutting tool) attached to the foreend thereof, the drill having a plurality of very hard abrasive grains 22 fixed thereto.
(b) The one shown in FIG. 4 which is composed of a wire 24 and a functional member 25 formed at its foreend (which serves as a cutting tool to collect a portion of living tissues or to remove occlusive matter), as disclosed in JP 1994(06)-189965 A.
(c) The one shown in FIG. 5 which has an end effecter 26 (as a cutting tool) attached to the foreend thereof, as disclosed in WO 2003/088833.
(d) The one shown in FIG. 6 which is composed of a shaft and a rotary member 29 (as a cutting tool) attached to the foreend thereof, the cutting tool having a spiral projection 27 or a blade 28, as disclosed in JP 2004-16504 A.
(e) The one shown in FIG. 7 which is an endoscope provided with the cable 31 having the screw 30 (as a cutting tool) attached to its foreend, as disclosed in JP 1989(01)-131641 A.

Each of the foregoing examples of the long tubular body is characterized by its structure and function as follows.

(a) The drill 23 is formed from stainless steel, titanium alloy, Ni—Ti shape memory alloy (Nitinol), or biocompatible plastic material having sufficient strength and stiffness. The drill 23 has very hard abrasive grains 22 (such as diamond abrasive grains) of 1 to 50 μm in diameter fixed to its foreend by electrodeposition, as a cutting tool. This drill 23 moves back and forth in response to the reciprocating movement of the motor 20 placed at the base end of the long tubular body, so that it makes a hole in the bone head.

(b) The wire 24 should preferably be formed from any unrestricted metallic materials which have no adverse effects on the human body, such as stainless steel, titanium alloy, Ni—Ti shape memory alloy (Nitinol), or biocompatible plastic materials with sufficient strength and stiffness. Also, the functional member 25 to be attached to the foreend of the wire 24 should preferably be formed from any unrestricted materials (such as cold-drawn wire of austenite stainless steel having elongated fibrous texture for reinforcement) excelling in strength and having no adverse effects on the human body. The functional member 25 should preferably be smaller in maximum diameter than the wire 24. The functional member 25 shown in FIG. 4A, which serves as a cutting tool of the medical instrument, is not restricted in structure so long as it is capable of drilling the bone head. Examples of the functional member 25 include the one shown in FIG. 4B which has a plurality of thorns formed around the circumference thereof, the one shown in FIG. 4C which has spiral projecting blades formed around the circumference thereof, the one shown in FIG. 4D which has a plurality of blade-like projections spirally arranged around the circumference thereof, and the ones shown in FIGS. 4E to 4G which have the spherical part at the foreend of the functional member shown in FIGS. 4B to 4D. The functional member 25 drills the bone head as it rotates while moving back and forth.

(c) The end effecter 26 should preferably be formed from any unrestricted metallic materials which have no adverse effects on the human body, such as stainless steel, titanium alloy, Ni—Ti shape memory alloy (Nitinol), or biocompatible plastic materials with sufficient strength and stiffness. The end effecter 26 drills the bone head by rotation and translational movement.

(d) The rotary member 29 (shaft) has a conically pointed tip which facilitates insertion. Moreover, it has spiral projections 27 around the circumference thereof whose edges are rounded to prevent damage to the tissue in contact with them. That part of the spiral projections 27 which is close to the pointed tip has a sharp cutting edge 28 smaller in outside diameter than the spiral projections 27. Upon rotation in the clockwise direction, the rotary member 29 constructed as mentioned above removes the bone tissue in contact with the sharp cutting edge 28, thereby making a hole. Conversely, upon rotation in the counterclockwise direction, the rotary member 29 allows the outside of the cutting edge 28 to smoothly come into contact with the bone tissue without cutting (or without making a hole).

(e) The screw 30 should preferably be formed from any unrestricted metallic materials which have no adverse effects on the human body, such as stainless steel, titanium alloy, Ni—Ti shape memory alloy (Nitinol), or biocompatible plastic materials with sufficient strength and stiffness. The screw 30 drills the bone head as it rotates and projects.

The long tubular body used in the present invention may employ laser as the cutting unit. In this case, it is composed of a guide wire and a guiding catheter (both for introduction close to the entrance of the retinaculum artery) and a light source for laser. The laser for this purpose is not specifically restricted; any known one may be used as such or with proper modifications. Examples include the laser described in JP 3467268 B and JP 4340834 B and the eximer laser catheter for angioplasty (made by DVX Co., Ltd.).

The long tubular body is not restricted in any other structure, such as the number of the lumen, diameter, and length, and the presence or absence of balloon. They should be properly determined by the clinician in consideration of the thickness of the artery and the ease with which it is transferred to the desired part.

The long tubular body having the cutting part at its foreend may be any commercial one. Its examples include the rotablator for ablation of calcified blood vessels (“Rotablator Advancer/Catheter” from Boston Scientific Japan Co., Ltd.).

The long tubular body may be made partly radiopaque at the shaft or the cutting part (particularly the foreend of the cutting part), so that the operator can confirm that the catheter or the foreend of the cutting part has been placed as the desired position. It is desirable that a contrast marker be attached to the cutting part, particularly the foreend of the cutting part. The contrast marker is not specifically restricted so long as it is formed from a radiopaque substance or any known contrast medium selected from iodine, barium, bismuth, boron, bromine, calcium, gold, platinum, silver, iron, manganese, nickel, gadolinium, dysprosium, tungsten, tantalum, stainless steel, Nitinol, and compounds thereof such as barium sulfate. Any contrast marker is acceptable so long as it permits the operator to confirm the position of the cutting part in the bone. In the case where the cutting part contains plastics material, the amount of the contrast marker should be 5 to 70 wt % of the plastic material. Alternatively, the foreend of the cutting part may be formed from a radiopaque metallic material.

The long tubular body may also be modified such that it delivers a contrast medium from its foreend, thereby allowing the operator to confirm the position of the foreend of the guide wire or to confirm that the catheter or the foreend of the cutting part has been placed at the desired position. This makes it possible to start drilling at the desired position. In other words, the method according to the present invention should preferably be applied in such a way that a contrast medium is injected through the long tubular body so that the operator can confirm that the foreend of the long tubular body has been placed at the desired position (the vicinity of the entrance of the retinaculum artery) before the operator starts drilling up to the bone head beyond the epiphysis line of the femoral head. Here, the contrast medium is not specifically restricted so long as it is a radiopaque substance selected from any known contrast media such as water-soluble iodine contrast medium (e.g., nonionic water-soluble iodine contrast medium), hyposmotic water-soluble iodine contrast medium, and such substance (and compounds thereof) as iodine, barium, bismuth, boron, bromine, calcium, gold, platinum, silver, iron, manganese, nickel, gadolinium, dysprosium, tungsten, tantalum, stainless steel, Nitinol, and barium sulfate, and solution or dispersion thereof. The amount of the contrast medium is usually 1 to 10 mL, which is sufficient for the operator to confirm that the catheter has been placed at the desired position in the artery.

Step (ii) is intended to perform drilling, by using the cutting tool 6 attached to the foreend of the long tubular body 1, up to the bone head, preferably up to the bone head beyond the epiphysis line 7 of the femoral head, as shown in FIGS. 1D and 2D. According to one embodiment, drilling in the bone head is accomplished in the following way. First, the long tubular body 1, which has the cutting tool 6 at its foreend, is placed in the retinaculum artery 3 in Step (i) as shown in FIG. 1C. Then, the long tubular body 1 is provided with a rotary unit (not shown) at its base. The rotary unit turns the cutting tool 6 at 100 to 400,000 rpm, preferably 1,000 to 300,000 rpm, more preferably 2,000 to 200,000 rpm. With the cutting tool 6 turning, the long tubular body 1 is advanced for drilling in the bone head, as shown in FIG. 1D. To be more specific, drilling is accomplished in the following way. First, the long tubular body 1, which has the cutting tool 6 at its foreend, is placed in the retinaculum artery in Step (i) as shown in FIG. 1C. Then, the long tubular body 1 is provided with a rotary unit (not shown) at its base. The rotary unit turns the cutting tool 6 at 1000 rpm. With the cutting tool 6 turning, the long tubular body 1 is advanced for drilling in the bone head, as shown in FIG. 1D. The distance of advancement is about 2 to 4 cm from the vicinity of the entrance of the retinaculum artery up to the point 1 to 10 mm inside the foreend of the bone head beyond the epiphysis line 7. After drilling, the long tubular body is withdrawn to the vicinity of the entrance 4 of the retinaculum artery 3. If necessary, the long tubular body 1 is placed in another retinaculum artery 3′ and drilling in the bone head is accomplished by using the cutting tool 6 in the same way as mentioned above, as shown in FIG. 1E. The foregoing procedure forms a plurality of perfusion passages 8 for blood which extend from the vicinity of the entrance of the retinaculum artery 3 to the bone head beyond the epiphysis line 7, as shown in FIG. 1F. After the foregoing procedure is completed, the long tubular body 1, the microcatheter 10′, and the guiding catheter 10 are withdrawn, and hemostasis is performed on that part from which they have been inserted and withdrawn. According to another embodiment, drilling in the bone head is accomplished in the following way. First, the long tubular body 1, which has the cutting tool 6 at its foreend, is placed in the retinaculum artery 3 in Step (i) as shown in FIG. 2C. Then, the long tubular body 1 is provided with a rotary unit (not shown) at its base. The rotary unit turns the cutting tool 6 at 100 to 400,000 rpm, preferably 1,000 to 300,000 rpm, more preferably 2,000 to 200,000 rpm. With the cutting tool 6 turning, the long tubular body 1 is advanced for drilling in the bone head, as shown in FIG. 2D. After drilling, the long tubular body is withdrawn to the vicinity of the entrance 4 of the retinaculum artery 3. The foregoing procedure forms the perfusion passage 8 for blood which extends from the branch point (entrance) 4 of the circumflex artery 5 and the retinaculum artery 3 to the bone head beyond the epiphysis line 7, as shown in FIG. 2D. To be more specific, drilling is accomplished in the following way. First, the long tubular body 1, which has the cutting tool 6 at its foreend, is placed in the retinaculum artery in Step (i) as shown in FIG. 2C. Then, the long tubular body 1 is provided with a rotary unit (not shown) at its base. The rotary unit turns the cutting tool 6 at 10,000 rpm. With the cutting tool 6 (or the foreend) turning at 10,000 rpm, the long tubular body 1 is advanced for drilling in the bone head, as shown in FIG. 2D. The distance of advancement is about 2 to 4 cm from the vicinity of the entrance 4 of the retinaculum artery 3 up to the point 1 to 10 mm inside the foreend of the bone head beyond the epiphysis line 7. After drilling, the long tubular body is withdrawn to the vicinity of the entrance 4 of the retinaculum artery 3. If necessary, the long tubular body is placed in another retinaculum artery 3′ and drilling in the bone head is accomplished by using the cutting tool 6 in the same way as mentioned above, as shown in FIG. 2E. The foregoing procedure forms a plurality of perfusion passages 8 for blood which extend from the vicinity of the entrance 4 of the retinaculum artery 3 to the bone head beyond the epiphysis line 7, as shown in FIG. 2F. After the foregoing procedure is completed, the long tubular body 1 and the guiding catheter 10 are withdrawn, and hemostasis is performed on that part from which they have been inserted and withdrawn. Incidentally, in the foregoing embodiments, the cutting tool 6 may be replaced by laser. The perfusion passage receives blood flowing therein from the retinaculum artery, the circumflex artery, and blood vessels in the ligament in contact with the bone head. The blood flowing in the perfusion passage releases the angiogenesis factor (stem cells derived from the bone marrow) into the perfusion passage. The thus released angiogenesis factor (stem cells derived from the bone marrow) forms blood vessels (capillary vessels) in the perfusion passage, thereby improving the blood flow (or eliminating ischemia) in the femoral head suffering from ischemia on account of bone head necrosis. The method according to the present invention is effective for the early patient who shows the symptom of bone head necrosis before the bone head is crushed because the crushed bone is not completely recovered after the restoration of the blood flow. In addition, the method according to the present invention does not involve surgical operation and hence is little invasive to the patient.

In Step (ii), no restrictions are imposed on the position of drilling by the cutting tool so long as drilling reaches the bone head beyond the epiphysis line, but drilling should not cross the foreend of the bone head. There is a distance of about 5 cm between the vicinity of the entrance of the retinaculum artery and the foreend of the bone head, and there is a distance of about 1.8 cm between the end of the retinaculum artery and the foreend of the bone head. With this point taken into consideration, the perfusion passage 8 should be so formed as to extend from the vicinity of the entrance 4 of the retinaculum artery 3 toward the point which is about 1 to 30 mm, preferably about 2 to 10 mm, inside the foreend of the bone head beyond the epiphysis line 7 of the femoral head. The perfusion passage is not specifically restricted in size. Its size should be properly selected according to the diameter of the blood vessel (capillary vessel) to be induced by the angiogenesis factor. Usually, the diameter of the perfusion passage should be 0.3 to 2 mm, preferably 0.5 to 1 mm. Incidentally, the size of the perfusion passage should be substantially equal to the diameter of the cutting tool attached to the foreend of the long tubular body. Consequently, it is desirable that the diameter of the cutting tool attached to the foreend of the long tubular body be substantially equal to the preferable range of the diameter of the perfusion passage. In addition, in Step (ii), it is possible to confirm the position of drilling by the cutting tool (or whether the foreend of the cutting tool has crossed the epiphysis line of the femoral head) by the distance of advance of the long tubular body or by X-ray radioscopy. Incidentally, the foregoing embodiment has been illustrated with reference to the long tubular body shown in FIG. 4A (which has the cutting tool as shown in FIG. 4B. However, the method according to the present invention is not restricted to the long tubular body shown in the foregoing embodiment. The long tubular body mentioned above may be replaced by any one having the cutting tool of the different structure as mentioned above or any one of the long tubular body of the different structure having the cutting tool.

This step (ii) should preferably be repeated to form a plurality of perfusion passages for effective improvement in blood flow in the bone head over a wider range. In other words, it is desirable to make more than one perfusion passage from the vicinity of the entrance of the retinaculum artery in the spongy bone of the bone head beyond the epiphysis line, thereby promoting the flow of blood into the bone head. Here, the number of perfusion passages to be formed is not specifically restricted, but it should be properly selected according to the type and graveness of bone head necrosis and the condition of the patient. To be more specific, 1 to 10, preferably 2 to 5 perfusion passages, should be formed from the entrance of one retinaculum artery (or the branch point of the circumflex artery and the retinaculum artery) in the bone head.

Step (ii) may also be modified as follows by substitution for or addition to what has been mentioned above. That is, it is desirable to place the long tubular body having the cutting tool at its foreend in the vicinity of one to five entrances (or the branch point of the circumflex artery and the retinaculum artery) of the retinaculum artery, and form at least one perfusion passage, as shown in FIGS. 1F and 2F. This procedure effectively improves the blood flow over a wider range in the bone head. However, drilling should be performed in such a way as not to cause fracture.

Step (ii) mentioned above may be carried out while injecting through the long tubular body 1 a vasodilator, a drug to promote vascularization, or cells derived from human tissues. Alternatively, it is also possible to inject a vasodilator, a drug to promote vascularization, or cells derived from human tissues through the long tubular body 1 which is being withdrawn after drilling to the prescribed position. A vasodilator, a drug to promote vascularization, or cells derived from human tissues which has been injected into the newly formed perfusion passage effectively forms blood vessels (capillary vessels). The injected cells differentiate into cells of blood vessels and bones, thereby inducing the vascularization in the perfusion passage and the restoration of necrotized parts. In other words, the above-mentioned drilling with the cutting tool in the bone head should preferably be followed by injection of a vasodilator, a drug to promote vascularization, or cells derived from human tissues through the long tubular body 1, thereby promoting the flow of blood. In this case, the long tubular body has two openings each at the far end and the near end and also has a lumen for liquid delivery to the far end. Any method may be employed for injection of a vasodilator, a drug to promote vascularization, or cells derived from human tissues. One way is by connecting a syringe containing a vasodilator to the hub at the base and injecting it into the perfusion passage from the foreend or side of the long tubular body during or after drilling. Incidentally, the base end of the long tubular body may be separated from the syringe by a three-way stopcock placed between them. Alternatively, the injection of a vasodilator into the perfusion passage may be accomplished by means of a pump from a bag containing a vasodilator connected to the hub at the base end of the long tubular body. In this way it is possible to inject a vasodilator slowly at a constant rate.

The vasodilator to be used in the foregoing step is not specifically restricted, and it should be properly selected according to the type and graveness of bone head necrosis and the condition of the patient. To be concrete, it includes the following. Prostaglandin, prostaglandin derivative, nonsteroidal anti-inflammatory drug (NSAID), steroidal anti-inflammatory drug, antiplatelet drug, anticoagulant, vitamins, muscle relaxant, antidepressant, poly-ADP-ribosepolymerase (PARP), excitatory amino acid receptor antagonist, radical scavenger, astrocyte function improver, IL-8 receptor antagonist, immunosuppressor, vascular growth factor, aldose reductase inhibitor, phosphodiesterase (PDE) inhibitor, and nitroglycerin and cardiac stimulant containing it. Preferable among them are prostaglandin, prostaglandin derivative, nonsteroidal anti-inflammatory drug (NSAID), steroidal anti-inflammatory drug, antiplatelet drug, vitamins, muscle relaxant, antidepressant, poly-ADP-ribosepolymerase (PARP), excitatory amino acid acceptor antagonist, radical scavenger, astrocyte function improver, IL-8 acceptor antagonist, and immunosuppressor.

Examples of prostaglandin include the following without restrictions. Prostaglandin A₁, prostaglandin A₂, prostaglandin A₃, prostaglandin B₁, prostaglandin B₂, prostaglandin B₃, prostaglandin C₁, prostaglandin C₂, prostaglandin C₃, prostaglandin D₁, prostaglandin D₂, prostaglandin D₃, prostaglandin E₁, prostaglandin E₂, 8-isoprostaglandin E₂, prostaglandin E₃, prostaglandin F_{1α}, prostaglandin F_{2α}, 13,14-dihydro-15-keto-prostograndin F_{2α}, 8-isoprostaglandin F_{2α}, 8-iso-13,14-dihydro-15-keto-prostoglandin F_{2α}, 8-epiprostaglandin F_{2α}, prostaglandin F_{3α}, prostaglandin G₁, prostaglandin G₂, prostaglandin G₃, prostaglandin H₁, prostaglandin H₂, prostaglandin H₃, prostaglandin I₁, prostaglandin I₂, prostaglandin I₃, prostaglandin J₂, 6-keto-prostaglandin F_{1α}, 2,3-dinor-6-keto-prostaglandin F_{1α}, 13,14-dihydro-15-keto-prostaglandin E₂, 7α-hydroxy-5,11-diketo-tetranor-prosta-1,16-dionic acid, and 5α,7α-dihydroxy-11-keto-tetranor-prosta-1,16-dioninc acid. The above-mentioned prostaglandin may be used as such or in the form of free base or salt. Examples of prostaglandin in the form of salt include the following without restrictions. Salt of alkali metal (such as potassium and sodium), salt of alkaline earth metal (such as calcium and magnesium), ammonium salt, pharmaceutically acceptable organic amine (such as tetramethylammonium, triethylamine, methylamine, dimethylamine, cyclopentylamine, benzylamine, phenetylamine, pyperidine, monoethanolamine, diethanolamine, tris(hydroxymethyl)aminomethane, lysine, arginine, N-methyl-D-glucamine, and acid adduct (such as salt of inorganic acid, including hydrochloride, hydrobromide, hydroiodide, sulfate, phosphate, and nitrate; and salt of organic acid, including acetate, lactate, tartrate, benzoate, citrate, methanesulfonate, ethanesulfonate, benzenesulfonate, toluenesulfonate, isethionate, glucuronate, and gluconate).

Examples of the preparation of prostaglandin derivative include the following without restrictions. Alprostadil, ornoprostil, limaprost, gemeprost, beraprost, trimoprostil, misoprostol, arbaprostil, and enprostil.

Examples of nonsteroidal anti-inflammatory drug (NSAID) include the following without restrictions. Sasapyrine, sodium salicylate, aspirin, aspirin-dialuminate, diflunisal, indometacin, suprofen, ufenamate, dimethylisopropylazulene, bufexamac, felbinac, diclofenac, tolmetin sodium, clinoril, fenbufen, nabumetone, proglumetacin, indometacin farnesil, acemetacin, proglumetacin maleate, amfenac sodium, mofezolac, etodolac, ibuprofen, ibuprofen piconal, naproxen, flurbiprofen, flurbiprofen axetil, ketoprofen, fenoprofen calcium, tiaprofen, oxaprozin, pranoprofen, loxoprofen sodium, aluminoprofen, zaltoprofen, mefenamic acid, aluminum mefenamate, tolfenamic acid, floctafenine, ketophenylbutazone, oxyphenbutazone, piroxicam, tenoxicam, ampiroxicam, napageln ointment, epirizole, tiaramide hydrochloride, tinoridine hydrochloride, emorfazone, sulpyrine, migrenin, saridon, sedes G, amipiro-N, sorbon, pilin cold medicine, acetaminophen, phenacetin, dimetotiazine mesilate, simetride-containing drug, and non-pyrine cold remedy.

Examples of antiplatelet drug include the following without restrictions. Aspirin, ticlopidine, ticlopidine hydrochloride, clopidogrel, dipyridamole, cilostzol, ozagrel, ozagrel sodium, prasugrel, ethyl icosapentate, beraprost, sarpogrelate, sarpogreleate hydrochloride, limaprost, GPIIb/IIIa receptor antagonist (such as abciximab), tirofiban, eptifibatide, and YMO28), AZD6140, and beraprost sodium.

Examples of anticoagulant include the following without restrictions. Heparin, warfarin, acenocoumarol, phenindione, citric acid, and EDTA.

Examples of vitamins include the following without restrictions. Vitamin B₁ and vitamin B₁₂.

Examples of muscle relaxant include the following without restrictions. Tolperisone hydrochloride, chlorzoxazone, chlormezanone, methocarbamol, phenprobamate, pridinol mesilate, chlorphenesin carbamate, baclofen, eperisone hydrochloride, afloqualone, tizanidine hydrochloride, alcuronium chloride, suxamethonium chloride, tubocurarine chloride, dantrolene sodium, pancuronium bromide, and vecuronium bromide.

Examples of antidepressant include the following without restrictions. Imipramine hydrochloride, desipramine hydrochloride, clomipramine hydrochloride, trimipramine maleate, amitriptyline hydrochloride, nortriptyline hydrochloride, lofepramine hydrochloride, amoxapine, and dosulepin hydrochloride, which are of tricyclic type; and maprotiline and mianserin, which are of tetracyclic type.

Examples of poly-ADP-ribosepolymerase (PARP) include the following without restrictions. 1,5-dihydroxyisoquinoline.

Examples of excitatory amino acid receptor antagonist include the following without restrictions. NMDA receptor antagonist and AMPA receptor antagonist.

Examples of radical scavenger include the following without restrictions. Edaravone and ebselen (DR-3305).

Examples of astrocyte function improver include the following without restrictions. ONO-2506.

As for IL-8 receptor antagonist, any known IL-8 receptor antagonist can be used without restrictions.

Examples of immunosuppressor, include the following without restrictions. Ciclosporin and FK506.

Examples of vascular growth factor include the following without restrictions. HIF (Hypoxia Inducible Factor), FGF (Fibroblast Growth Factor), and PDGF (Platelet-Derived Growth Factor).

Examples of aldose reductase inhibitor include the following without restrictions. Epalrestat, fidarestat, AS-3201, zenarestat, imirestat, AL-4114, ICI-10552, ICI-215918, ZD-5522, BAL-ARI8, methosorbinil, FR-62765, WF-2421, GP-1447, IDD-598, JTT-811, ADN-138, ADN-311, lindolrestat, SG-210, M-16049, M-16209, NZ-314, sorbinil, zopolrestat, CP-AR-3192, ascorbyl gamolenate, risarestat, salfredins, AD-5467, TJN-732, TAT, tolrestat, thizocin-A, axillarin, ICI-215918, ponalrestat, minalrestat, DN-108, SPR-210, and A-74863a.

Examples of phosphodiesterase (PDE) inhibitor include the following without restrictions. PDE3 inhibitor, PDE4 inhibitor, and PDE5 inhibitor. PDE3 inhibitor is exemplified by aminone, milrinone, vesnarinone, cilostazol, and sildenafil. PDE4 inhibitor is exemplified by Cilomilast (Ariflo®), Roflumilast (BY-217), Arofylline, OPC-6535, ONO-6126, IC-485, AWD-12-281, CC-10004, CC-1088, KW-4490, Lirimilast, ZK-117137, YM-976, BY-61-9987, CC-7085, CDC-998, MEM-1414, ND-1251, Bay 19-8004, D-4396, PD-168787, Atizoram (CP-80633), Cipamfylline (BRL-61063), Rolipram, NIK-616, SCH-351591, and V-11294A. PDE5 inhibitor is exemplified by Sildenafil and Sildenafil citrate. Other PDE inhibitors include NT-702.

Examples of the drug to promote vascularization may be selected unrestrictedly from any known vascular inducers which include the following. Vascular growth factor, such as VEGF121, VEGF165, and VEGF189; vascular endothelium growth factor (VEGF) family (described in J. Pathol. 1998; 184(1): 53-57); fibroblast growth factor (FGF) family (described in Cell Biol. International 1995: 19(5): 431-444, and JACC 1993; 7:2001-2006); transforming growth factor (TGF)-α and -β (described in Surg. Neurol. 1998; 49(2): 189-195); platelet-derived growth factor (PDGF) (described in Proc. Natl. Acad. Sci. 1990; 87:2628-2632, Annu. Rev. Cell Dev. Biol. 1995; 11:73-91, and Cancer Res. 1997; 57:963-969); and Ang-1 and Ang-2.

The human tissue-derived cells include without restrictions marrow, peripheral blood, synovial membrane, and cord blood and any other collectable cells derived from tissues. They are exemplified by vascular endothelium precursor cells, marrow-derived mesenchymal stem cells, mononuclear cells, platelet-rich plasma (PRP), platelet-poor plasma (PPP), fibrin clot, concentrated growth factor (CGF), synovial membrane-derived stem cells, cord blood-derived stem cells and precursor cells, fat-derived stem cells, nerve stem cells, pancreas stem cells, amnion-derived stem cells, blood-forming stem cells, liver stem cells, pulp stem cells, sperm stem cells, cornea-derived stem cells, cuticle-derived stem cells, hair follicle stem cells, iPS cells, ES cells, Muse cells, osteoblast, osteoclast, cartilage cells, fibroblast, other stem cells, tissue cells, and derived cells. Collected tissues are separated into components capable of induction into bones and blood vessels (which are suitable for infusion) without treatment or after such treatment as concentration, separation, and culture of cells. This process is accomplished by, for example, collecting bone marrow liquid from the ilium or femur of an anesthetized patient and stem cells derived from bone marrow are separated by using a separator such as SmartPReP2 BMAC (Harvest Technologies Inc.). The separated cells are filled into a syringe and then infused into the drilled part through the long tubular body. Infusion may be assisted by mixing cells with a scaffold material such as gel, an artificial bone that functions as a footing for cells, a component that increases viscosity, and a component that induces the differentiation of cells. Examples of the footing for cells include the following without restrictions. Collagen, elastin, agarose, alginate, synthetic peptide, fibroin, fibrin, hyaluronic acid, hyaluronic benzyl ester, alginic acid, calcium alginate hydrogel, chondroitin sulfate, heparan, keratan, PRP, hydrogel, gelatin, fibronectin, laminin, vitronectin, tenascin, thrombospondin, heparin, polylactic acid (PLA), polyglycolic acid (PGA), hydroxyapatite, β-tricalcium phosphate (TCP), and temperature-responsive polymers. Examples of the component to increase viscosity include the following. Magnesium, calcium, hyaluronic acid, chondroitin sulfate, and contrast medium. Examples of the component that induces the differentiation of cells include the following. Blood vessel growth factor, blood vessel induction factor, BMP family, IGF family, Sox family, Wnt family, GATA family, Bcl family, TNF family, IL family, cAMP, Notch signal, insulin, testosterone, parathyroid hormone, estradiol, growth hormone, angiopoetin family, retinoic acid, vitamin, dexamethasone, miRNA, and differentiation induction inhibitor factor antigen.

Preferable among the foregoing examples are low-molecular weight collagen, PRP, hyaluronic acid, and low-viscosity readily flowable hydrogel, which are capable of easy infusion through the catheter. Other desirable ones are fibrin (immediately after mixing with fibrinogen and thrombin) and any polymer that changes in viscosity with temperature and hardens after infusion. A typical example is a temperature-responsive hydrogel of hyaluronic acid, as disclosed in Japanese Patent Application No. 2005-512109.

The vasodilator drug, the drug to promote angiogenesis, and the human tissue-derived cells, which have been mentioned above, may be used alone or in combination with one another. In addition, the vasodilator or the drug to promote angiogenesis may be used in the form of sustained release preparations for injection into the perfusion passage so that it is supplied continuously. This type of preparations may also be used for mixture with the human tissue-derived cells. Examples of the sustained release preparations include the following without restrictions. Microcapsule preparations, microsphere preparations, and nanosphere preparation (all for injection), and the above-mentioned scaffold material. Any ordinary sustained release injection drugs may be used, preferably in the form of microcapsule, microsphere, and nanosphere. The microcapsule, microsphere, and nanosphere preparations are defined as those preparations which contain the above-mentioned vasodilator or drug to promote angiogenesis as an active ingredient and take on the form of fine particles in combination with a bioabsorbable or biodegradable polymer.

The controlled drug release system that employs the foregoing sustained release preparations allows the vasodilator or the drug to promote angiogenesis to produce its effect within the bone head over a long period of time, thereby inducing angiogenesis. The foregoing sustained drug release system may employ a bioabsorbable polymer or a biodegradable polymer which is either a natural polymer or a synthetic polymer. The rate of sustained release may be controlled by decomposition, diffusion, or membrane permeation.

Here, the examples of the natural polymer as a bioabsorbable polymer include the following without restrictions. Vegetable-produced polysaccharides (such as cellulose, starch, and alginic acid), animal-produced polysaccharides and proteins (such as chitin, chitosan, collagen, gelatin, alubumin, and glycosaminoglycan), and microbe-produced polyester and polysaccharide (such as poly-3-hydroxyalkanoate and hyaluronic acid).

Examples of the biodegradable polymer include the following without restrictions. Fatty acid ester polymer or copolymer, polyacrylic ester, polyhydroxylactic acid, polyalkyleneoxalate, polyorthoester, polycarbonate, and polyaminoacid. They may be used alone or in combination with one another. The fatty acid ester polymer or copolymer is exemplified by polylactic acid, polyglycolic acid, polycitric acid, polymalic acid, polyethylene succinate, polybutylene succinate, poly-ε-caprolactone, polybutylene terephthalate-adipate, and lactic acid-glycolic acid copolymer. They may be used alone or in combination with one another. Additional examples include poly-α-cyanoacrylic ester, poly-β-hydroxylactic acid, polytrimethyleneoxalate, polyorthoester, polyorthocarbonate, polyethylene carbonate, poly-γ-benzyl-L-glutamic acid, polyvinyl alcohol, polyester carbonate, polyacid anhydride, polycianoacrylate, polyphosphazene, and poly-L-alanine, which may be used alone or in combination with one another. Preferable among these examples are polylactic acid, polyglycolic acid, and lactic acid-glycolic acid copolymer, and lactic acid-glycolic acid copolymer is most desirable. The biodegradable polymer is preferably one which has a weight-average molecular weight of about 2,000 to 800,000, more preferably about 5,000 to 200,000. For example, the polylactic acid preferably has a weight-average molecular weight of about 5,000 to 100,000, more preferably about 6,000 to 50,000. It may be synthesized by any known process. The lactic acid-glycolic acid copolymer is preferably composed of lactic acid and glycolic acid in a ratio of from about 100/0 to 50/50 (by weight), especially from about 90/10 to 50/50 (by weight). It preferably has a weight-average molecular weight of about 5,000 to 100,000, more preferably about 10,000 to 80,000. It may be synthesized by any known process. It may be incorporated with a basic amino acid (such as alginic acid) for prevention of initial burst. Incidentally, the weight-average molecular weight specified in the present invention is expressed in terms of the molecular weight of polystyrene determined by gel permeation chromatography (GPC). The dose of the biodegradable polymer may be properly varied according to the pharmacological activity of the active ingredient and the rate of release of the intended drug so long as it achieves the object of the present invention. For example, the ratio of the biodegradable polymer to the vasodilator is preferably about 0.2 to 10,000 times (by weight), more preferably about 1 to 1,000 times (by weight), and still more preferably about 1 to 100 times (by weight).

The microspheres, microcapsules, and nanocapsules may be produced unrestrictedly by any known process, such as the one disclosed in JP 2010-120964 A, with or without modifications. The known process specifically includes in-water drying method, (such as o/w method, w/o method, and w/o/w method), phase separation method, spray-dry method, and granulation method by supercritical fluid.

The sustained release preparations may be converted into the sustained release injection drug by any known process without restrictions, such as the one disclosed in JP 2010-120964 A, with or without modifications. For example, an injection drug of microspheres may be prepared by changing microspheres into an aqueous dispersion with the help of dispersing agent, preservative, tonicity adjusting agent, buffering agent, and pH adjustor. It is also possible to prepare an injection drug of microspheres in the form of oily suspension by dispersing microspheres together with vegetable oil, mixture of vegetable oil and phospholipid (such as licithin), or medium chain triglyceride (such as migriol 812). The microspheres are not specifically restricted in diameter. In the case where they are used for a suspension injection drug, their diameter should be small enough for dispersion and needle passage. An adequate diameter is preferably about 0.1 to 300 μm, more preferably about 1 to 150 μm, and still more preferably about 2 to 100 μm. Microspheres may be made into sterile preparations by any method, for example, by keeping the entire process sterile, by sterilization with γ-rays, or by adding antiseptics.

In the present invention, the vasodilator should be administered in an adequate dose that depends on the kind of the vasodilator, the type of preparations, the duration of drug release, the kind and graveness of the bone head necrosis, and the condition of the patient. An adequate dose for one time is about 0.5 to 10 μg, preferably about 1 to 5 μg, for an adult (weighing 50 kg).

The method mentioned above permits more than one perfusion passage to be formed in the bone head with the help of the long tubular body. The perfusion passage flows blood through it together with the vascular inductive factor (stem cells derived from marrow) which induces the formation of blood vessels (capillary vessels) in the perfusion passage, thereby improving the blood flow in the bone head. The method according to the present invention does not need surgical operation and hence it is low-invasive with a very small burden on the patient.

### EXAMPLES

The following is a detailed description of the embodiment of the method which is particularly suitable for transluminally delivering a long tubular body (cutting catheter or cutting wire) having a cutting tool at its foreend close to the entrance of the retinaculum artery and then drilling up to the femoral head beyond the epiphysis line of the femur. The scope of the present invention is not limited by the examples that follow.

### Example 1

A patient suffering from femoral head necrosis undergoes X-ray or MRI examination to ascertain the range of necrosis. At least 24 hours before insertion of a catheter, the patient is orally given clopidogrel (as an antiplatelet agent) (300 mg) once a day on the first day of administration. If antithrombotic treatment is necessary, the patient is orally given it (75 mg for maintenance dose) once a day at the same timing as above.

The patient has a sheath placed in the femoral artery of the other one of the patient's leg having a lesion, with the help of a catheter introducer kit (Radifocus Introducer, made by Terumo Corporation). Through this sheath is inserted a guide wire, 0.035 inches in diameter (Radifocus Guide Wire M, made by Terumo Corporation). The guide wire is advanced under X-ray radioscopy until its foreend reaches the inside femoral circumflex artery through the deep artery of thigh from the femoral artery. Along the guide wire is inserted a guiding catheter 4Fr (Radifocus catheter M for angiography, made by Terumo Corporation). This catheter has its foreend placed under X-ray radioscopy at the branch point of the inside femoral circumflex artery and the deep artery of thigh. When it is confirmed under X-radioscopy that the foreend of the catheter has been placed at the branch point of the inside femoral circumflex artery and the deep artery of thigh, the guide wire is withdrawn.

Through the guiding catheter is inserted a microcatheter (2.2 Fr) which holds a guide wire (0.014 inches in diameter) passing through it, and the guide wire is advanced. When the foreend of the microcatheter enters the retinaculum artery from the branch point of the inside femoral circumflex artery and the retinaculum artery, the guide wire is withdrawn. Through the microcatheter is inserted a cutting wire (0.014 inches) as shown in FIG. 4A, and the foreend of the cutting wire is placed in the retinaculum artery within the bone. To the base end of the cutting wire is connected a rotary drive unit, so that the cutting wire is turned at 1,000 rpm. The cutting wire is advanced under X-ray radioscopy to the point which is 2 cm away from the point of placement in the retinaculum artery and beyond the epiphysis line of the femoral head and 3 mm inside the foreend of the bone head. The base of the cutting catheter is twisted so that the cutting part is turned through about 30 degrees. The foregoing step is repeated five times, so as to form five perfusion passages in the bone head. After the foregoing treatment, the cutting wire and the guiding catheter are withdrawn and hemostasis is performed on the femoral artery.

### Example 2

A patient suffering from femoral head necrosis undergoes X-ray or MRI examination to ascertain the range of necrosis. At least 24 hours before insertion of a catheter, the patient is orally given clopidogrel (as an antiplatelet agent) (300 mg) once a day on the first day of administration. If antithrombotic treatment is necessary, the patient is orally given it (75 mg for maintenance dose) once a day at the same timing as above.

The patient has a sheath placed in the femoral artery of the other one of the patient's leg having a lesion, with the help of a catheter introducer kit (Radifocus Introducer, made by Terumo Corporation). Through this sheath is inserted a guide wire, 0.035 inches in diameter (Radifocus Guide Wire M, made by Terumo Corporation). The guide wire is advanced under X-ray radioscopy to the femoral artery where there exists the lesion and then inserted to the point which is slightly beyond the branch point of the deep artery of thigh and the outside femoral circumflex artery. Along the guide wire is inserted a guiding catheter 4Fr (Radifocus catheter M for angiography, made by Terumo Corporation). This catheter has its foreend placed under X-ray radioscopy at the branch point of the outside femoral circumflex artery and the deep artery of thigh. When it is confirmed under X-radioscopy that the foreend of the catheter has been placed at the branch point of the outside femoral circumflex artery and the deep artery of thigh, the guide wire is withdrawn.

Through the guiding catheter is inserted a rotablator (“Rotablator Advancer/Catheter” made by Boston Scientific Japan Co., Ltd.) which holds a guide wire (0.009 inches in diameter) passing through it, and the guide wire is preceded. When the foreend of the rotablator reaches the branch point of the outside femoral circumflex artery and the retinaculum artery, the guide wire is withdrawn. To the base end of the rotablator is connected a rotary drive unit, so that the rotablator is turned at 100,000 rpm. The rotablator is advanced under X-ray radioscopy to the point which is 4 cm away from the vicinity of the entrance of the retinaculum artery and beyond the epiphysis line of the femoral head and 3 mm inside the foreend of the bone head. The base of the rotablator is twisted so that the cutting tool is turned through about 30 degrees. The foregoing step is repeated five times, so as to form five perfusion passages in the bone head. After the foregoing treatment, the rotablator and the guiding catheter are withdrawn and hemostasis is performed on the femoral artery.

### Example 3

The same procedure as in Example 2 is repeated except that the cutting catheter is advanced to the point which is beyond the epyphysis line of the femoral head and 3 mm inside the foreend of the bone head, with slow injection from a syringe inserted into the catheter hub at the end of the rotablator. The syringe contains 0.5 mL of alprostadil as a vasodilator, and the rate of injection is 50 μL/min (or 250 ng/min of alprostadil).

### Example 4

The same procedure as in Example 1 is repeated except that the cutting wire shown in FIG. 4A is replaced by the 2.2Fr cutting catheter which holds therein a guide wire (0.014 inches in diameter) having a cutting tool (shown in FIG. 6) at the foreend thereof.

### Example 5

The same procedure as in Example 1 is repeated except that the cutting wire is advanced to the point which is beyond the epyphysis line of the femoral head and 10 mm inside the foreend of the bone head and then the cutting wire is withdrawn, and the procedure is completed by slowly injecting (through the microcatheter) 0.5 mL of parenteral solution containing 2.5 μg of alprostadil (prostaglandin derivative) as a vasodilator at a rate of 50 μL/min (or 250 ng/min for alprostadil).

### Example 6

The same procedure as in Example 1 is repeated except that the cutting wire is advanced to the point which is beyond the epyphysis line of the femoral head and 10 mm inside the foreend of the bone head and then the cutting wire is withdrawn, and the procedure is completed by injecting (through the microcatheter) marrow stem cells separated by using SmartRPep2 BMAC.

### Example 7

The same procedure as in Example 1 is repeated except that the cutting wire is advanced to the point which is beyond the epyphysis line of the femoral head and 10 mm inside the foreend of the bone head and then the cutting wire is withdrawn, and the procedure is completed by injecting (through the microcatheter) a mixture of marrow stem cells (separated by using SmartRPep2 BMAC) and a temperature-responsive polymer.

The present invention, without its scope being limited to the foregoing examples, may be applied to the long bone and the end thereof suffering from any other diseases than bone head necrosis, which typically include bone tumor, false joints, bone fracture, cartilage damage, osteomyelitis, osteonecrosis, spinal tumor, and hip joint disease.

What is claimed is:

1. A method for improving the blood flow in the bone head, said method comprising the steps of extending a long tubular body, which has a cutting tool at its foreend, close to the entrance of the retinaculum artery and performing drilling on the bone head by using said cutting tool.

2. The method for improving the blood flow in the bone head as defined in claim 1, which further comprises a step of performing antithrombotic treatment before drilling the bone head with said cutting tool, thereby preventing thrombi from occurring after the resumption of the blood flow.

3. The method for improving the blood flow in the bone head as defined in claim 1, which further comprises a step of drilling with said cutting tool at least one perfusion passage from the vicinity of the entrance of the retinaculum artery in the spongy bone of the bone head beyond the epiphysis line, so as to promote the blood flow into the bone head.

4. The method for improving the blood flow in the bone head as defined in claim 1, which further comprises a step of injecting a vasodilator, a drug to promote vascularization, or human tissue-derived cells through said long tubular body when a hole is drilled or after a hole has been drilled in the bone head by using said cutting tool, thereby promoting the perfusion of blood.

5. The method for improving the blood flow in the bone head as defined in claim 1, which further comprises a step of injecting a vasodilator, a drug to promote vascularization, or human tissue-derived cells mixed with at least one kind of scaffold material through said long tubular body when a hole is drilled or after a hole has been drilled in the bone head by using said cutting tool, thereby promoting the perfusion of blood.
